(19) United States
(12) Patent Application Publication
(10) Pub. No.: US 2012/0094371 A1
(43) Pub. Date: Apr. 19, 2012

(54) TEST STRIP FOR H. PYLORI DETECTION

(76) Inventors: Philip Ross, Tampa, FL (US); Morris Behar, Oldsmar, FL (US)

(21) Appl. No.: 13/275,386

(22) Filed: Oct. 18, 2011

Related U.S. Application Data
(60) Provisional application No. 61/394,451, filed on Oct. 19, 2010.

(51) Int. Cl. C12M 1/34

(52) U.S. Cl. 435/288.7

(57) ABSTRACT

A test device, including: a compartment defining an environment and comprising a specimen region; an indicator medium disposed in the compartment; a separation medium disposed in the compartment between the specimen region and the indicator medium; an active ingredient adjacent the specimen region; and a color indicator disposed on the indicator medium and responsive a chemical compound produced by a reaction involving the active ingredient by changing from a first color to a second color. The separation medium is configured to permit fluid communication directly between the environment and a side of the indicator medium proximate the separation medium when the separation medium and the indicator medium abut each other.

### CROSS-REFERENCE TO RELATED APPLICATIONS

This application claims the benefit of U.S. Provisional Application No. 61/394,451 filed Oct. 19, 2010, and incorporated herein by reference in its entirety.

### BACKGROUND OF THE INVENTION

This invention relates to testing and diagnostic devices suitable for diagnosing gastrointestinal disorders. Specifically, the invention provides a testing device for diagnosing gastrointestinal disorders by the detection of the urease enzyme.

Helicobacter pylori (H. pylori) is a type of bacterium that is responsible for many gastrointestinal disorders including chronic histological gastritis, peptic ulcers, non-ulcer dyspepsia, gastric cancers and gastroduodenal ulcers. By causing chronic inflammation H. pylori may weaken the mucosal defenses and allow acid and pepsin to disrupt the epithelium. H. pylori grows on the gastric epithelium, does not penetrate the tissues, and is protected from stomach acid by the action of a urease which the bacterium produces. The urease hydrolyzes endogenous urea from the host tissues to form the chemical compound ammonia which neutralizes stomach acid. H. pylori produces large amounts of urease enzyme which allows H. pylori to utilize urea as a nitrogen source. In addition, the breakdown of urea produces high local concentrations of ammonia which enables the organism to tolerate low pH according to the reaction below:

(NH₂)CO+2H₂O+H⁺→2NH₄⁺+HCO₃⁻

Prior art tests for detecting H. pylori include blood tests, ¹³C and ¹⁴C breath tests, biopsy of the gastric mucosa followed by histological culture and direct microscopic examination, and the urease enzyme test. Blood tests are non-invasive; however, they suffer from the drawbacks of creating a false positive due to a cross-reaction with other antibodies as well as the blood test remaining positive for many months after the bacteria have been eradicated. The ¹³C and ¹⁴C breath tests operate on the principle that if urea marked with ¹³C and ¹⁴C were ingested, it is transformed into marked CO₂ in the stomach if H. pylori is present and can be detected in exhaled air. The breath tests have the drawback of requiring the availability of expensive isotopes and expensive equipment to analyze the results which makes them infeasible for commercial use. Invasive tests such as histological culture and the urease enzyme test are more commonly used. However, the histology culture method is difficult and time consuming, thus it is not feasible to use this test commercially. Despite their expense, these tests are still used commercially. Those of ordinary skill in the art consider these testing methods to be less accurate and less sensitive than the urease enzyme test. Further, the previous tests suffer from drawbacks such as the bulkiness of the testing equipment, the use of an aqueous solution requiring the necessity to refrigerate the test kit, the length of the reaction time and the cost of the test.

The urease enzyme test is considered the most efficient test method to date. As stated previously, H. pylori produces a high degree of urease enzyme. Human tissues do not produce urease and if present, H. pylori is the principal urease producing microorganism inhabiting the gastric region. Generally, a biopsy of the gastric region is obtained, normally endoscopically, and is incubated in a medium containing urea and a pH sensitive dye. The urease produced by H. pylori releases ammonia when urea is hydrolyzed which increases the pH and thus changes the color of the pH indicator.

In the prior art urease enzyme test, a test device may include a compartment for holding a filter layer, an indicator layer, and a biopsy specimen. Within the compartment the indicator layer is placed against the compartment inner surface, the filter layer is placed adjacent the indicator layer, a biopsy specimen is placed on the filter layer, and the compartment is sealed.

The filter layer may contains an antibiotic or antimicrobial agent which may be used to help prevent bacterial growth, prolong the life of the test, and increase the specificity of the test by inhibiting the growth of other microorganisms that may contaminate the specimen. Examples of microbial agents include biocides such as sodium azide or potassium sorbate. The filter layer may also contain a buffering agent which assists in achieving the proper pH levels necessary to the urea-urease reaction. The initial pH is preferably about 6. An example of a buffering agent that may be used in the present exemplary embodiments is sodium dihydrogen phosphate. The filter layer may also contain urea as the active ingredient which is used to react with any urease in the biological sample. The reaction between urea and urease creates ammonia. A presence of ammonia changes the pH within the chamber.

The indicator layer may include an antimicrobial agent, a buffering agent, urea and a pH color indicator such as phenol red, which is used to detect the change in pH. The pH color indicator reacts to a change in pH caused by the ammonia and in the case of phenol red changes color from yellow to red. If such a color change occurs, the presence of ammonia is indicated, which indicates the presence of H. pylori in the specimen (positive result). If a color change is not seen, the test indicates a negative result for the presence of H. pylori. While phenol red is discussed, the pH color indicator used can be any of those known in the art.

In previous dry method tests for H. pylori, the indicator layer is a circular shaped disk with an inner void in the center making the indicator layer resemble a ring, a.k.a., an annulus. The indicator layer is covered by an integral water impervious barrier on a side adjacent the filter layer/biopsy specimen. This integral waterproof barrier is present in the prior art tests to prevent false indications for the presence of H. Pylori resulting from direct contact between the biopsy specimen and any of its fluids and the indicator layer, and any associated unwanted color change in the indicator layer. However, this integral waterproof barrier also prevents the ammonia from coming into direct contact with a proximal side of the indicator layer adjacent the filter layer (and the biopsy specimen). A distal side of the indicator layer is pressed against an inner surface of the compartment. As a result, the only surfaces of the indicator layer that are effectively exposed to the ammonia are an inner perimeter surface and an outer perimeter surface. Due to the configuration of the prior art test the compartment is transparent near the indicator layer distal side surface and, as such, it may be that only the indicator layer distal side surface is visible to the user. Thus, it is the indicator layer distal side surface that must change color to indicate and to confirm a positive indication.

While the indicator layer may begin to turn color in the regions of the indicator layer inner perimeter surface and the indicator layer outer perimeter surface when ammonia contacts those surfaces, which suggests a positive indication, color change limited to these areas is not sufficient to confirm a positive indication. Further indication may be required because blood or other contaminants on the biopsy specimen may color those surfaces in similar manner, which may result a false indication if only the initial coloring were relied upon.

In order for the ammonia to reach some or all of the indicator layer distal side surface, as may be required for a positive indication, ammonia must be absorbed into the indicator layer until it reaches some or all of the indicator layer distal side surface. However, the absorption process through the indicator layer is relatively slow. This is so because the ammonia can only be absorbed through the exposed indicator layer inner perimeter surface and the indicator layer outer perimeter surface. These exposed surfaces together account for only a small percentage of a total surface area of the indicator layer, and thus serve as a choke point for ammonia entering the indicator layer. Further, the ammonia must migrate so it can reach all radial locations along the indicator layer distal side surface. This means the ammonia may be required to 1) migrate from indicator layer inner perimeter surface, which is the most radially inward surface, all the way to the indicator layer outer perimeter surface, which is the most radially outward surface, or 2) vice versa, or most likely 3) two discrete ammonia migrations may be absorbed at each edge and migrate toward each other until they meet. In all cases the ammonia is required to migrate a relatively long distance through the indicator layer material. Likewise, ammonia may be slow to work its way between the indicator layer distal side surface and the inner surface of the compartment. This migration is particularly slow if the indicator layer is pressed hard against the inner surface of the compartment 30, which may happen when a biopsy specimen is on the large side, for example. These slow migration processes result in relatively slow indication using the prior art testing device.

### BRIEF DESCRIPTION OF THE INVENTION

A device for testing for the presence of H. pylori, an exemplary embodiment including: a compartment defining an environment and comprising a specimen region; an indicator medium disposed in the compartment; a separation medium disposed in the compartment between the specimen region and the indicator medium; an active ingredient adjacent the specimen region; and a color indicator disposed on the indicator medium and responsive a chemical compound produced by a reaction involving the active ingredient by changing from a first color to a second color. The separation medium is configured to permit fluid communication directly between the environment and a side of the indicator medium proximate the separation medium when the separation medium and the indicator medium abut each other.

A test device for testing for the presence of H. pylori, another exemplary embodiment including: a compartment defining an environment and having a specimen region; an indicator medium disposed in the compartment and having a side proximate the specimen region and a side distal from the specimen region; a separation medium disposed in the compartment between the specimen region and the indicator medium; a filter medium disposed between the specimen region and the separation medium; an active ingredient disposed on the filter medium; and a color indicator disposed on the distal side of the indicator medium and responsive to a chemical compound produced by a reaction involving the active ingredient by changing color. When the chemical compound is present and when the separation medium abuts the proximate side of the indicator medium at least a first portion of the chemical compound enters the proximate side of the indicator medium and migrates through the indicator medium before reaching the color indicator disposed on the distal side of the indicator medium.

### BRIEF DESCRIPTION OF THE DRAWINGS

For a fuller understanding of the invention, reference should be made to the following detailed description, taken in connection with the accompanying drawings, in which:

FIG. 1 is a front perspective view of an exemplary embodiment of the testing device.

FIG. 2 is an exploded view of an exemplary embodiment of the testing device illustrating the different layers present in the bulge.

FIG. 3 is a side view of an exemplary embodiment of the testing device illustrating the bulge protruding from beneath the support member.

FIGS. 4 and 5 are perspective views of exemplary embodiments of the separation layer.

### DETAILED DESCRIPTION OF THE INVENTION

In the following detailed description of the preferred exemplary embodiments, reference is made to the accompanying drawings, which form a part hereof, and within which are shown by way of illustration specific exemplary embodiments by which the invention may be practiced. It is to be understood that other exemplary embodiments by which the invention may be practiced. It is to be understood that other exemplary embodiments may be utilized and structural changes may be made without departing from the scope of the invention.

Exemplary embodiments disclosed herein address the drawbacks of the previous tests to provide an inexpensive dry method test strip having a reaction time of less than one hour.

As shown collectively in FIGS. 1-5, exemplary embodiments provide a test strip 10 which may include a support member 20, a cover sheet 50, a filter layer 90, a separation layer 80, an indicator layer 70 and a sealing cap 60. (As used here the terms layer and medium may be used interchangeably.) The support member 20 has a first 22 and a second side 24. A compartment 30 is disposed within the support member 20 for holding the filter layer 90, separating layer 80 and indicator layer 70 as well as the biopsy specimen (not shown) in a specimen region 64 (shown approximately). The compartment 30 is preferably circular in shape; however other shapes may be used. The opening of the compartment 30 lies flush with the first side 22 of the support member 20 and a hemispherical dome 31 may protrude from the second side 24 of the support member 20. The support member 20 may be manufactured of any rigid material such as glass or a clear plastic. While it is preferable that the support member 20 is transparent, the support member 20 may be opaque as long as the hemispherical dome 31 of the compartment 30 is transparent to allow the user to analyze the results of the reaction.

The support member 20 can be substantially rectangular in shape with one end 26 of the support member 20 having a notch 28 for ease of grasping the cover sheet 50. The support member 20 may also contain a draining channel 40, which may be etched in or cut completely through the first side 22 of the support member 20 to allow excess liquid from the biopsy specimen, or excess air within the compartment 30, to drain. In some exemplary embodiments, the portion 52 of the cover sheet 50 exposed by the notch 28 in the support member 20 can prevent contaminants from entering the compartment 30 through the draining channel 40. Alternatively or in addition, the portion of the cover sheet 50 exposed by the notch can be treated with a desiccant 54 to prevent moisture from entering the compartment 30 through the draining channel 40. Likewise, desiccant 54 can be disposed anywhere proximate the draining channel 40, so long as it is effective to reduce an amount of moisture entering the compartment 30 through the draining channel 40.

The cover sheet 50 is generally flexible and is comprised of a first side 56 and a second side 58. The second side 58 of the cover sheet 50 is preferably adhesively attached to the first side 22 of the support member 20. The cover sheet 50 may extend an entire length of the support member 20 covering the first side 22 of the support member 20. Alternately, the cover sheet 50 may only cover as much of the first side 22 of the support member 20 as is necessary. The cover sheet 50 may have a sealing cap 60 that may be integral to the cover sheet 50, may be attached to the first side 56 of the cover sheet 50, or may be a plastic cap that seals the compartment without the use of an adhesive sheet by a snap fit, or other fit known to those of ordinary skill in the art. The cap may further be attached to the support member 20 by a tether. The sealing cap 60 is positioned so as to be in alignment with the compartment 30 disposed within the support member 20. The sealing cap 60 may be of the same shape as the compartment 30 and may be sized so as to fit within the compartment 30 to provide a tight seal to the compartment 30. The sealing cap 60 may contain a slight ridge 62 along the perimeter to provide a tighter seal to the compartment 30. The sealing cap 60 may be manufactured of a rigid or semi-rigid plastic.

As depicted in FIG. 2, the compartment 30 may be used to house several different layers. The first layer may be the filter layer 90. This layer may be generally circular or disk-shaped and of sufficient dimensions so as to easily fit within the compartment 30. The filter layer 90 may be the outermost layer which lies closest to the cover sheet 50 (as shown in FIG. 1) and/or the sealing cap 60 (as shown in FIG. 1). The biopsy specimen is placed on the filter layer 90 after which the sealing cap 60 covers the compartment 30 to seal the specimen into the compartment 30. This helps prevent the ammonia from escaping and the biopsy specimen and the test components from drying out. The filter layer 90 may be manufactured of a filter paper such as the filter paper made by Whatman (Paper Grade 4); however, one of ordinary skill in the art would be able to readily determine other filter papers that could be used.

The filter layer 90 may contains an antibiotic or antimicrobial agent which may be used to help prevent bacterial growth, prolong the life of the test, and increase the specificity of the test by inhibiting the growth of other microorganisms that may contaminate the specimen. Examples of microbial agents include biocides such as sodium azide or potassium sorbate. The filter layer 90 may also contain a buffering agent which assists in achieving the proper pH levels necessary to the urea-urease reaction. The initial pH may be preferably about 6. An example of a buffering agent that may be used in an exemplary embodiment is sodium dihydrogen phosphate. However, other buffering agents may be used as are known to those of ordinary skill in the art. The filter layer 90 may also contain urea as the active ingredient which is used to react with the urease in the biological sample. The reaction between urea and urease creates an ammonium ion which causes a change in pH. This change in pH caused by contact with ammonia in turn causes the color of the pH indicator in the indicator layer 70 to change thus confirming the presence of H. pylori. Ammonia is referenced herein as the chemical compound that causes the change of color in the pH indicator. However, while the structure disclosed herein may be used with the chemical elements disclosed herein, it may also be used with any substitute equivalent chemicals that function in the same way. The filter layer 90 may contain coloring, preferably yellow, to provide a contrast to the indicator layer 70 in the event that H. pylori is present and the indicator layer 70 changes color. The exemplary compounds contained in the filter layer 90 are not meant to be exclusive and one of ordinary skill in the art would be capable of identifying similar compounds that would be within the scope of the exemplary embodiments. The concentrations of the compounds are those which are therapeutically acceptable.

The middle layer is a separation layer 80 which is disposed between the filter layer 90 and the indicator layer 70. The separation layer 80 may be a perforated layer having the same dimensions as the filter layer 90 and the indicator layer 70 and proportioned to be contained within the compartment 30 of the support member 20. When urease in the biological sample comes into contact with urea in the filter layer 90, ammonia is formed. When the ammonia comes into contact with the pH indicator present on the indicator layer 70, the pH indicator changes color. With respect to a location of the biopsy specimen and/or the separation layer 80, the indicator layer 70 may have an indicator layer proximate side surface 72, an indicator layer distal side surface 74, an indicator layer inner perimeter surface 76, and an indicator layer outer perimeter surface 78.

The separation layer 80 disclosed herein represents an improvement to previous dry urease test methods in that the separation layer 80 permits a configuration that enables the indicator layer proximate side surface 72 to come into direct contact with the ammonia generated by the reaction between urea and urease while still eliminating the possibility for false indications produced by physical contact between the filter layer and the indicator layer 70. As a result of this physical separation of the indicator layer 70 from the biopsy specimen, which is a source for material that might produce false positives, the need for the water impervious layer integral to the indicator layer 70 of previous tests is eliminated.

The indicator layer 70 is extremely thin and any ammonia absorbed by the indicator layer proximate side surface 72 will almost immediately migrate through the indicator layer 70 to the indicator layer distal side surface 74. Once the ammonia has migrated through the very thin indicator layer 70, the migrated ammonia will cause the pH indicator on the indicator layer distal side surface 74 to change color. Since the migration through the relatively extremely thin indicator layer 70 occurs much faster than the radial migrations of the prior art test, the change in color of the indicator layer distal side surface 74, and thus the indication of the present device occur significantly faster. Significantly faster indication represents an advantage to those awaiting for the indication.

Further, instances where there is a relatively small amount of H. Pylori present in the biopsy specimen, scenarios may exist where some ammonia is produced, but it is not enough to migrate sufficiently throughout the indicator layer 70 within the prescribed time period in the prior art testing devices. However, with the instant device, since the time required to migrate through the indicator layer 70 is essentially negligible, it is more likely that the color indicator will change color within the prescribed time frame. Thus the instant device decreases the chances of a false negative reading.

Still further, since the gases no longer must enter the indicator layer 70 via the inner perimeter surface 76 in the exemplary embodiments, the need for the inner perimeter surface 76 itself is eliminated. Consequently, indicator layer 70 may be shaped without the inner void 71. This represents an improvement because without the inner void 71, a total surface area of the indicator layer distal side surface 74 increase from that of an annulus to that of a circle. An increase in the surface area that changes color increases the visibility of a color change, thereby making the indication more visible than the prior art tests.

So long as the separation layer also enables direct contact of the ammonia with some or all the indicator layer proximate side surface 72, any configuration for the separation layer 80 is acceptable. In an exemplary embodiment, the separation layer 80 may be permeable to the ammonia, or the separation layer 80 may be manufactured of any type of netting or mesh, either rigid or flexible. An example of a type of mesh that can be used in the present exemplary embodiments is a nylon mesh. The thickness of the separation layer 80 as well as the mesh hole size can be readily determined by those of ordinary skill in the art. Using a separation layer 80 including mesh accomplishes the requisite separation of the prior art, yet enables direct exposure of the indicator layer proximate side surface 72 to the ammonia which, in turn, expedites the indication process.

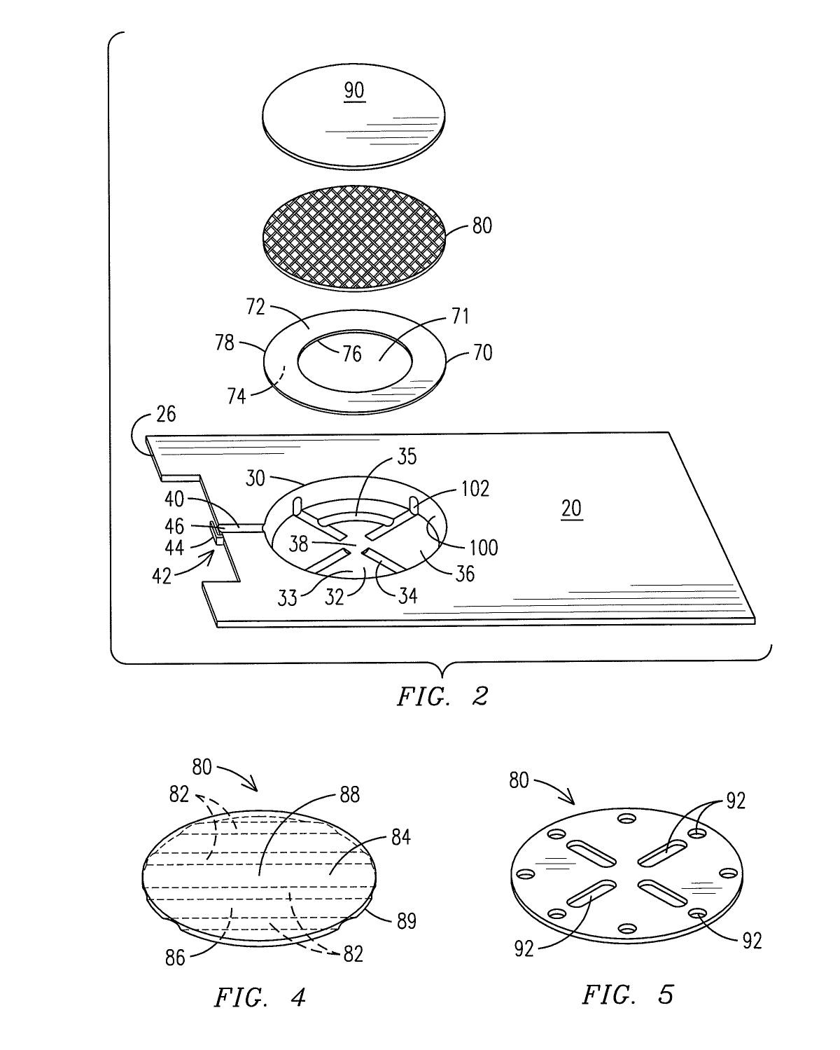

Other configurations for the separation layer may be considered. For example, as shown in FIG. 4, the separation layer 80 having a separation layer proximal side 84 and a separation layer distal side 86, may instead have no holes there through, but may have channels 82 on the separation layer distal side 86. The channels 82 may be further configured to permit ammonia to travel to a separation layer inner region 88 from a separation layer outer edge 89, and therefore across the across the indicator layer proximate side surface. Alternately, as can be seen in FIG. 5, the separation layer 80 may be a solid disc with apertures 92 of various sizes and shapes and in various locations.

Referring back to FIG. 2, the innermost layer may be the indicator layer 70. This layer may be disposed on a side of the separation layer 80 opposite the filter layer 90 and biopsy specimen, and may be in contact with the inner surface 32 of the compartment 30, and the inner surface 32 may be transparent. An end surface 33 portion of the inner surface 32 of the compartment 30 may comprise recesses such as end radial grooves 34 in an exemplary embodiment with a circular compartment 30, or any other shape effective to enable ammonia to reach the indicator layer distal side surface 74 directly in exemplary embodiments with a compartment 30 with a shape other than circular. In an exemplary embodiment the end radial grooves 34 may be oriented in an array such that ammonia may be able to travel between an outer region 36 of the compartment 30 and an inner region 38 of the compartment 30, and respective regions of the indicator layer distal side surface 74 indicator layer proximate side surface 72 more easily. In an exemplary embodiment where the indicator layer 70 comprises an inner void 71, the ammonia may reach the end radial grooves 34 may receive ammonia via the inner void 71. In addition, the end surface 33 may comprise end circumferential grooves 35 that may be in fluid communication with the end radial grooves 34, and these may also enable direct contact of the ammonia with additional regions of the indicator layer distal side surface 74, expediting the indication process still further.

A side wall 100 portion of the inside surface of the compartment 30 may likewise have recesses, such as side wall grooves 102. The side wall grooves 102 walls may extend partially or fully along a length of the side wall 100, and may be configured to align with and therefore communicate ammonia to the end radial grooves 34. In such an exemplary embodiment, the ammonia may by free to travel in either direction in the end radial grooves 34, and likewise in either direction across the indicator layer distal side surface 74. Alternately, the side wall grooves 102 may not align with the end radial grooves 34. The recesses may take on any shape in addition to the groove shape illustrated.

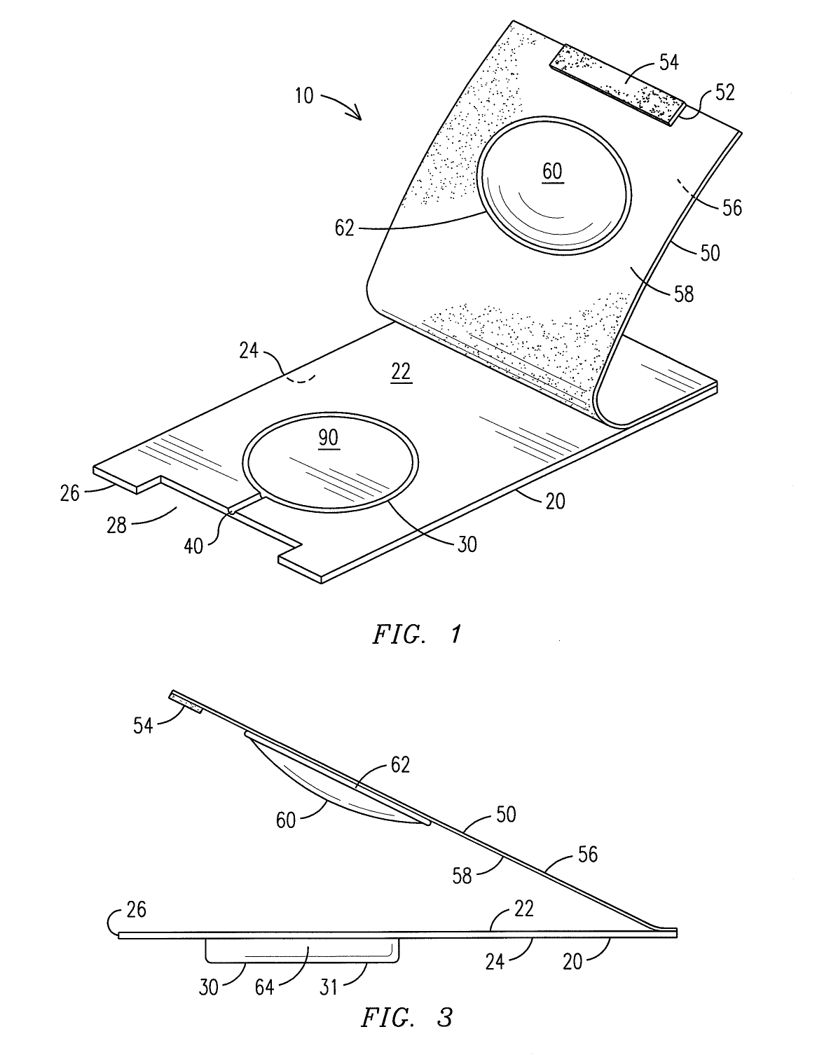

As seen collectively in FIGS. 2 and 3, compartment 30 may protrude from the second side 24 of the support member 20. The indicator layer 70 may be sandwiched between the inner surface 32 of the hemispherical dome 31 of the compartment 30 and the separation layer 80. Similar to the filter layer 90 and separation layers 90, the indicator layer 70 is of sufficient dimension and thickness to be contained within the compartment 30. The indicator layer 70 may be circular in shape and may or may not have a circular void 71 in the middle.

The indicator layer 70 may contain an antimicrobial or antibiotic agents such as sodium azide or potassium sorbate. Similar to the antimicrobial agent used in the filter layer 90, the antimicrobial agent used in the indicator layer 70 acts to prevent bacterial growth and prolong the life of the test.

The indicator layer 70 also contains a pH color indicator, such as phenol red, which is used to detect the change in pH caused by the reaction of urea and urease. However, the pH color indicator used can be any of those known in the art provided the color indicator is capable of changing the color of the surfaces 72, 74, 76, 78 of the indicator layer 70. Preferably the pH color indicator has a pKa of between about 6.5 to about 8.5 which acts to sense pH change. Phenol red is known to have characteristics of appearing yellow in an acidic solution and a purplish-red color in a basic solution. In exemplary embodiment where an inner void 71 is present the indicator layer 70 may also change to a different color than the color of the separation layer 80. This enables a user to see the color change of the indicator layer 70 better via the contrast of the new color of the indicator layer 70 and the color of the separation layer 80. Alternately, in an exemplary embodiment where an inner void 71 is present and when the separation layer 80 is of a configuration that enables a user to see the filter layer 90 through the separation layer 80, the indicator layer 70 may change to a color that is different from the color of the filter layer 90.

Similar to the filter layer 90, the indicator layer 70 also contains a buffering agent such as sodium dihydrogen phosphate which assists in achieving the proper pH levels essential to the urea-urease reaction. The initial pH level is about 6. The indicator layer 70 may contain urea as the active component of the reaction between urea and urease to produce the ammonia. As with the filter layer 90, the exemplary compounds contained in the indicator layer 70 are not meant to be exclusive and one of ordinary skill in the art would be capable of identifying similar compounds that would be within the scope of the exemplary embodiments. The concentrations of the compounds are those which are therapeutically acceptable.

FIG. 3 is an image illustrating an exemplary embodiment where the hemispherical dome 31 of the compartment 30 protrudes from the second side of the support member 20. In exemplary embodiments where the indicator strip is not to be removed from the compartment 30 for viewing the indication, the hemispherical dome 31 of compartment 30 may be transparent to allow the user to examine the results of the urea-urease reaction. The layers may be disposed into the compartment 30 in the following order: the indicator layer 70 in contact with the hemispherical dome 31 of the compartment 30; the separation layer 80 positioned between the indicator layer 70 and the filter layer 90; and the filter layer 90 in contact with the separation layer 80. The biopsy specimen is positioned on the filter layer 90 and the sealing cap 60 may be inserted into the compartment 30 to provide a tight seal to the compartment 30 which prevents the specimen from drying out, or when integral to the cover sheet 50, the sealing cap 60 may be applied as the cover sheet 50 is applied. The draining channel 40 may be provided in the support member 20 to allow any excess fluid to drain out of the compartment 30 after the sealing cap 60 is in place. Fluids include liquids such as any that might be present from the biopsy specimen, and any gases, such as those generated by the test reaction, or air that may be trapped in the compartment 60.

FIG. 3 also illustrates the sealing cap 60 which may protrude from the second side of the cover sheet 50. The sealing cap 60 may be positioned to align with the opening of the compartment 30 in the first side of the support member 20. The sealing cap 60 may be of sufficient shape, size and dimension so as to fit within the compartment 30 and provide a tight seal when the compartment 30 is closed. The sealing cap 60 may further have a ridge 62 around its perimeter to enable a tighter seal when placed in the compartment 30. The sealing cap 60 serves to seal the test components, the biopsy specimen, and the ammonia within the compartment 30 and prevent it from drying out while the reaction is taking place.

In operation, a biopsy specimen is obtained from an individual. The biopsy specimen should be of an area of normal-looking tissue since an area containing ulcerations or erosions is likely to have smaller numbers of H. pylori present. The biopsy specimen should be of sufficient size, preferably 2-3 mm in diameter. The cover sheet 50 may be separate, or in an exemplary embodiment the cover sheet 50 may be peeled back from the support member 20 exposing the filter layer 90 contained within the compartment 30. The biopsy specimen is placed on the filter layer 90 and positioned so as to have maximum contact with the filter layer 90. The cover sheet 50 is then adhered, or in an exemplary embodiment re-adhered to the support member 20 with the sealing cap 60 positioned to seal the biopsy specimen within the compartment 30. Slight pressure may be applied to the cover sheet 50 and/or the sealing cap 60 to ensure that a tight seal is formed between the sealing cap 60 and the compartment 30. This slight pressure allows any excess fluid such as from the biopsy specimen or trapped air to drain out of the compartment 30 via the draining channel 40. The pressure further allows the fluids from the biopsy specimen to make better contact with the filter layer. Since any urease present will be present in the fluids of the biopsy specimen, better contact between the filter layer containing the urea and the biopsy specimen fluids containing the urease will improve the ammonia generating reaction. Accurate sealing of the cover sheet 50 and sealing cap 60 to the support member 20 is important to ensure the biopsy specimen does not dry up. In an exemplary embodiment the draining channel 40 may be disposed in other locations of the support member 20. The draining channel 40 may further comprise a check valve 42. This may help prevent or slow any drying of the biopsy specimen. The check valve 42 may comprise a resilient member 44 disposed on the support member 20 proximate an outlet 46 of the draining channel 40, span the outlet 46, and be biased to permit fluids to escape, but not reenter the compartment 30. For example, the check valve could simply be a piece of material such as paper, or an equivalent, that acts like a leaf check valve or a reed valve across the outlet 46.

In interpreting the results, when the biopsy specimen is initially placed on the filter layer 90, the indicator layer 70 may have a pinkish tinge if blood or alkaline bile is present. The color of the tinge should be noted and the test strip should be re-examined in about 5 minutes. If the area has deepened in color and expanded in size any time after 5 minutes and up to an hour, the test is read as being positive for H. pylori. It is recommended that the test strip 10 is examined at intervals of 5 minutes, 30 minutes and one hour. If there is no initial pinkish tinge and the indicator layer 70 changes color from a yellow color to a red color, the test is positive for H. pylori. If there is an initial pinkish tinge, but the indicator layer 70 continues to change color, the test is positive for H. pylori. If H. pylori is present, the indicator layer 70 will gradually turn from yellow in color to a deep orange and finally a red color. A red color observed at the end of an hour's time is a positive reaction. If alternatively, the indicator layer 70 is still yellow in color after an hour's time, the biopsy specimen is negative for H. pylori. Only the indicator layer 70 changes color. The filter layer 90 does not contain a pH color indicator and thus the color of the filter layer 90 can be used as a reference for any color change in the indicator layer 70.

False positive reactions due to bacteria other than H. pylori will not normally react prior to 3 hours because these bacteria produce much less urease than H. pylori.

The disclosures of all publications cited above are expressly incorporated herein by reference, each in its entirety, to the same extent as if each were incorporated by reference individually.

It is also to be understood that the following claims are intended to cover all of the generic and specific features of the exemplary embodiments herein described, and all statements of the scope of the exemplary embodiments which, as a matter of language, might be said to fall there between. Now that the exemplary embodiments have been described,

What is claimed is:

1. A test device, comprising:
a compartment defining an environment and comprising a specimen region;
an indicator medium disposed in the compartment;
a separation medium disposed in the compartment between the specimen region and the indicator medium;
an active ingredient adjacent the specimen region; and
a color indicator disposed on the indicator medium and responsive a chemical compound produced by a reaction involving the active ingredient by changing from a first color to a second color,
wherein the separation medium is configured to permit fluid communication directly between the environment and a side of the indicator medium proximate the separation medium when the separation medium and the indicator medium abut each other.

2. The test device of claim 1, wherein the active ingredient comprises urea, the chemical compound produced by the reaction involving the urea comprises ammonia, and the color indicator is a pH color indicator responsive to a presence of ammonia.

3. The test device of claim 1, wherein the separation medium comprises mesh, or at least one opening, or at least one channel on a side abutting the indicator medium.

4. The test device of claim 1, comprising a filter medium disposed between the specimen region and the separation medium, and the active ingredient is disposed on the filter medium.

5. The test device of claim 1, wherein the compartment comprises an inside end surface adjacent a distal side of the indicator medium with respect to the specimen region, and wherein the compartment inside end surface comprises an end recess configured to enable fluid communication across at least a portion of the distal side of the indicator medium.

6. The test device of claim 5, wherein the compartment comprises an inside side surface adjacent a perimeter of the indicator medium, and wherein the compartment inside side surface comprises a side recess configured to enable fluid communication from a proximate side of the indicator medium to the distal side of the indicator medium.

7. The test device of claim 6, wherein the end recess and the side recess are in fluid communication with each other.

8. The test device of claim 1, comprising a sealing cap configured to seal the specimen region, the separation medium, and the indicator medium within the compartment.

9. The test device of claim 1, wherein the compartment is associated with a support member configured to support the compartment.

10. The test device of claim 9, comprising an adhesive cover sheet disposed on the support member and configured to span at least the compartment.

11. The test device of claim 10, wherein the adhesive cover sheet comprises an integral sealing cap configured to seal the specimen region, the separation medium, and the indicator medium within the compartment.

12. The test device of claim 11, comprising a drain channel configured to enable fluid communication between the compartment and an outside environment.

13. The test device of claim 12, comprising a check valve associated with the drain channel and configured to allow fluid to escape but not enter the compartment.

14. The test device of claim 12, comprising a desiccant disposed proximate the drain channel.

15. A test device, comprising:
a compartment defining an environment and comprising a specimen region;
an indicator medium disposed in the compartment comprising a side proximate the specimen region and a side distal from the specimen region;
a separation medium disposed in the compartment between the specimen region and the indicator medium;
a filter medium disposed between the specimen region and the separation medium;
an active ingredient disposed on the filter medium; and
a color indicator disposed at least on the distal side of the indicator medium and responsive to a chemical compound produced by a reaction involving the active ingredient by changing color,
wherein when the chemical compound is present and when the separation medium abuts the proximate side of the indicator medium at least a first portion of the chemical compound enters the proximate side of the indicator medium and migrates through the indicator medium before reaching the color indicator disposed on the distal side of the indicator medium.

16. The test device of claim 15, wherein the indicator medium comprises an annular shape, wherein the compartment comprises an inside side surface and an inside end surface, wherein the indicator medium distal side abuts the inside end surface, and wherein the inside end surface comprises an end recess configured to permit communication of a second portion of the chemical compound across the distal side of the indicator medium.

17. The test device of claim 16, wherein the inside side surface comprises a side recess configured to permit communication of the second portion of the chemical compound to the end recess.

18. The test device of claim 15, wherein the separation medium is permeable to the chemical compound.

19. The test device of claim 15, comprising a support member configured to support the compartment, and a flexible sealing cap configured to seal the specimen region and the color indicator within the compartment.

20. The test device of claim 19, comprising a drainage channel configured to enable fluid communication between the compartment and an outside environment, and a check valve associated with the drainage channel and configured to allow fluid to escape but not enter the compartment
